(19) United States
(12) Patent Application Publication
(10) Pub. No.: US 2023/0288410 A1
(43) Pub. Date: Sep. 14, 2023

(54) SYSTEMS AND METHODS FOR DETECTING CYTOKINES

(71) Applicant: University of Houston System, Houston, TX (US)

(72) Inventors: Chandra Mohan, Houston, TX (US); Rongwei Lei, Houston, TX (US)

(21) Appl. No.: 18/121,554

(22) Filed: Mar. 14, 2023

Related U.S. Application Data
(60) Provisional application No. 63/269,281, filed on Mar. 14, 2022.

(51) Int. Cl. G01N 33/543; G01N 33/68

(52) U.S. Cl. CPC G01N 33/54386; G01N 33/6869; G01N 2333/5412; G01N 2470/04

(57) ABSTRACT

Provided herein are vertical flow assay devices for detecting presence or an amount of cytokines in a sample. Also provided herein are immunoassay methods for detecting presence or an amount of cytokines in a sample, using the vertical flow assay devices.

### TECHNICAL FIELD

The disclosure relates to systems and methods for detecting cytokines in a sample.

### BACKGROUND

Cytokine detection is an important step in medical diagnosis, risk assessment, disease monitoring, and evaluation of treatment. For example, interleukin 6 (IL-6) IL-6 is a pleiotropic cytokine with broad-ranging effects within the integrated immune response. IL-6 levels in samples can support the presence, absence, or extent of various diseases and conditions associated with inflammation or infection, for example, rheumatoid arthritis, COVID-19 infection, and response to cancer treatment. A common way to detect a cytokine, such as IL-6, is an enzyme linked immunosorbent assay (ELISA). However, ELISA requires hours to days for the completion, which is not suitable for use in a rapid test for routine monitoring at clinic or at home. Accordingly, there is a need for systems and methods for detection of a cytokine in a biological sample.

### SUMMARY

The present disclosure provides systems, devices, and methods for determining presence and/or level of a cytokine in a biological sample. These systems, devices, and methods facilitate sensitive, rapid, and inexpensive testing and can handle a large volume capacity. Embodiments include systems and methods for detecting cytokines, such as IL-6, in a sample.

Certain embodiments include a vertical flow assay device for detecting presence or an amount of a cytokine in a sample. One such device contains (a) a membrane member having a first surface and a second surface, (b) a capture agent capable of specific binding to the cytokine and immobilized on or within the membrane member, (c) an absorbent pad member having a first surface and a second surface, and located under the membrane member, wherein the second surface of the membrane member and the first surface of the absorbent pad member are in contact with each other, (d) a holder with a first wall and a second wall each having a first surface and a second surface. The first wall of the holder is positioned proximal to the membrane member, such that the second surface of the first wall and the first surface of the membrane member are proximal to each other. This first wall has a cavity or opening that extends from the first surface to the second surface of the first wall of the holder. The opening traverses the first wall to expose a portion of the first surface of the membrane member to outside of the vertical flow assay device. The second wall of the holder is proximal to the absorbent pad member, such that the second surface of the absorbent pad member and the first surface of the second wall are proximal to each other. The holder is a tight-fit configuration that provides a chamber to contain the membrane member and the absorbent pad member.

In some embodiments, the membrane member of the vertical flow assay device is made of one or more porous solid state materials. These materials can be one or more of plastic, polymers, paper, nitrocellulose, cellulose, PVDF, polycarbonate, ceramic, metallic materials, glass, glass microfibers, and anodized aluminum. In some embodiments, the membrane member is a nitrocellulose membrane. In some embodiments, the membrane member has pores that are 10 micrometers (μm) or less in size and a porosity of between 10% and 95%. In certain embodiments, the membrane member has pores that are about 0.2 μm to about 0.5 μm in size.

In some embodiments, the absorbent pad member of the vertical flow assay device is made of one or more absorbent pads. In some embodiments, the absorbent pad member has pores that are between 1 μm and 1000 μm in size, and are spatially arranged to provide a substantially uniform flow rate profile across the membrane member. In certain embodiments, the absorbent pad member has thickness of between 1 mm to 10 mm at 53 kilopascal (kPa). In some embodiments, the absorbent pad member has thickness of about 4.7 mm at 53 kPa.

In some embodiments, the sample is a body fluid sample, such as a serum sample. In some embodiments, detection of presence or an amount of cytokine, such as IL-6, in a sample is facilitated by the formation of a sandwich complex. This sandwich complex includes a capture agent, a cytokine (if present), and a detection agent that binds to the cytokine. Also provided in certain embodiments is a detectable signal that is related to the presence or amount of the cytokine in the sample bound in the sandwich complex. In some embodiments, the detection agent is bound to a signal development element. In some embodiments, the signal development element contains gold nanoparticles. In certain embodiments, the gold nanoparticles are about 40 nanometers (nm) in diameter. In some embodiments, the capture agent and/or the detection agent is an antibody that binds to a cytokine, such as an anti-IL-6 antibody. In other embodiments, the detection agent can be a chromogenic detection agent, a fluorogenic detection agent, an enzymatic detection agent, or an electrochemiluminescent detection agent. In other embodiments, the capture agent and/or the detection agent can be an aptamer, receptor, affibody, mimic, nucleic acid, or a synthetic compound.

In some embodiments, the holder is a standard fitting configured to mount to a detection system, wherein a portion of the membrane member can be accessed through the cavity in the first wall of the holder to provide a detectable signal indicative of the presence or the amount of cytokine to the detection system. In some embodiments, the detection system includes an imager, a scanner, or a smartphone. In some embodiments, the vertical flow assay device further contains an additional cavity or opening for accessing a positive control and/or a negative control present on the membrane member. In some embodiments, the vertical flow assay device is disposable.

In some embodiments, the cytokine that is analyzed is IL-6. The minimal detectable concentration of IL-6 in the sample can be about 3.2 picograms per milliliter (pg/ml) or less.

Certain embodiments include immunoassay methods for detecting presence or an amount of a cytokine in a sample. One such method includes the steps of providing the vertical flow assay device previously described, applying a sample onto the membrane member through the cavity or opening of the first wall of the holder, applying a detection agent that binds to a cytokine, such as IL-6 (if present), thereby forming a sandwich complex containing the capture agent, a cytokine, and the detection agent. The method also includes the steps of providing a detectable signal that is related to the presence or amount of a cytokine in the sample bound in the sandwich complex, and relating the detectable signal to the presence or the amount of a cytokine in the sample.

Certain embodiments include immunoassay methods for detecting presence or an amount of a cytokine in a sample. One such method includes the steps of performing a vertical flow assay on the sample with a capture agent and a detection agent which together form a sandwich complex with a cytokine in a vertical flow assay device, wherein the immunoassay provides a detectable signal that is related to the presence or amount ofa cytokine in the sample bound in the sandwich complex. The method further includes relating the detectable signal to the presence or the amount of a cytokine in the sample. In some embodiments, the sample is a body fluid sample. In some embodiments, the detection agent is bound to a signal development element. In some embodiments, the signal development element contains gold nanoparticles. In some embodiments, the gold nanoparticles are about 40 nm in diameter. In some embodiments, the cytokine that is analyzed is IL-6 and the capture agent and/or the detection agent is an antibody that binds to IL-6.

In some embodiments, the immunoassay method further includes the steps of mounting the vertical flow assay device to a detection system to provide the signal, and determining the presence or amount of a cytokine in the sample. In some embodiments, the detection system includes an imager, a scanner, or a smartphone. In some embodiments, the cytokine that is analyzed is IL-6 and the detectable concentration of IL-6 in the sample is about 3.2 pg/ml or less. In some embodiments, the reportable range of IL-6 in the sample is from about 10 pg/ml or less to about 1,000 pg/ml or more. In some embodiments, the vertical flow assay device is disposable. In some embodiments, the presence or absence of a cytokine, such as IL-6, in the sample is provided within about 15 minutes from the contact of the sample on the membrane member.

Certain embodiments include a kit for detecting presence or an amount of a cytokine containing one or more of a vertical flow assay device of the present disclosure, a capture reagent, and a detection reagent that each bind to a cytokine, a signal development element that can bind to the detection reagent, a washing buffer, a blocking buffer, a calibration marker to relate a signal to the presence or the amount of a cytokine, and/or an instruction for use.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIG. 1 depicts an aerial view of an exemplary vertical flow assay system for detecting IL-6, according to the embodiments of the present disclosure.

FIG. 2 depicts certain components and assembly of a vertical flow assay system for detecting IL-6, according to the embodiments of the present disclosure.

FIGS. 3A-3C depict embodiments of methods of operation and assay interpretation.

FIG. 3A is a diagrammatic representation of certain assay steps of a method. FIG. 3B is a set of photographic images of a vertical-flow paper-based assay for IL-6 detection. Depicted is the workflow for assaying human serum IL-6 using the vertical-flow paper-based assay. In this embodiment, the paper-based assays are prepared by stacking two layers of 0.2 μm nitrocellulose membrane (NCM), one layer of Whatman® CF7 pad, and two layers of Whatman® CF5 pad. This combination of NCM and absorbent pads is clamped together to secure them into the plastic housing. The workflow starts with a wash, followed by adding blocking buffer, loading the sample, adding detection antibody, and then adding gold nanoparticles (GNP). There can be a wash step in between each of the addition steps. The signal is observed and quantified after the final wash. FIG. 3C is a set of representative images of a semi-quantitative determination of IL-6 using three reference standards. Shown is a set of six schematics of the IL-6 vertical flow assays, each demonstrating a test zone (top left quadrant) and three reference zones loaded with different concentrations of IL-6 standard. The observer score (OS) from 0 to +5 are indicated at the top of each image to show how this semi-quantitative score was assigned based on the relative intensities seen at the test zone and three reference zones included.

FIGS. 4A-4B depict effect of membrane pore sizes and layers on assay performance. In FIG. 4A, the two rows depict the seven types of porous membranes that were evaluated for detection of 100 pg/mL rIL-6 in 10-fold-diluted serum and non-spiked 10-fold diluted serum control. FIG. 4B is a graphical representation of the colorimetric intensity values as calculated using Image J and the signal over noise ratio (SNR) was computed for each type of membrane. The error bar indicates the standard deviation of three measurements of SNR.

FIGS. 5A-5B depict a dose response curve for the vertical flow paper-based assays with IL-6 spiked into human healthy serum using the vertical-flow paper-based assay. FIG. 5A is a set of representative images of the assay results using two layers of Amersham 0.2 μm membrane. Two replicates were obtained at each concentration and three replicates for the negative. The top left of the membrane represents the test zone. FIG. 5B is a graphical representation of the colorimetric intensity values calculated using ImageJ. The 3*σ+negative mean line indicates the summation of mean intensity of non-spiked healthy serum samples (0 pg/ml) and 3× their standard deviation (σ). The concentration above this line marks the limit of detection, which is 3.2 pg/ml. The dose response curve demonstrated a reportable range from 3.2 pg/ml to 10,000 pg/ml of IL-6 in ten-fold diluted serum.

FIGS. 6A-6C depict the reproducibility performance of the vertical flow assay (VFA).

FIG. 6A is a set of representative images of results of serial dilutions performed by three researchers. IL-6 VFA sets 1, 2, and 3 were performed by the same researcher so that the intra-operator CV for the standard curves can be computed. IL-6 VFA sets 1, 4, and 5 were performed by three different researchers, one per set, to determine the inter-operator CV. The membrane type displayed in FIG. 6A is Amersham 0.2 μm with two layers. FIGS. 6B-6C are graphical representations of the corresponding dose-response curves for computing intra-operator reproducibility (FIG. 6B) and inter-operator reproducibility (FIG. 6C). The dotted lines correspond to a value that equals the negative healthy serum mean plus three times the standard deviation. The tables associated with each curve show the inter-operator and intra-operator CV at each point concentration and average overall CV for the VFA assay. The relatively low inter- and intra-operator CV values indicate the assay results are reproducible across multiple researchers and also when repeatedly performed by a single researcher.

FIGS. 7A-7B depict the impact of storage on IL-6 VFA performance. FIG. 7A is a set of representative images of IL-6 VFA tests stored at room temperature for one day, two weeks, four weeks, and six weeks were tested for detection of varying doses of IL-6 spiked into buffer using Amersham 0.2 μm membrane with two layers. FIG. 7B is a graphical representation of the correlation of IL-6 level to the intensity on the VFA tests over different storage durations.

FIG. 8 is a graphical representation of the specificity of the rIL-6 VFA. Addition of glucose, creatinine, and glycine to rIL-6-spiked buffer did not significantly affect the intensity of the signal at the test zone compared to rIL-6-spiked buffer. IP-10, IL-8, and TNF-α spiked buffer showed significantly lower intensities than rIL-6-spiked buffer on the IL-6 VFA and similar intensities to the non-spiked buffer, indicating that the-VFA is relatively specific for IL-6 detection. Samples were loaded onto the VFA in triplicates. ^{ns} p>0.05, **** p<0.0001 compared to the positive control, as determined using one-way ANOVA.

FIGS. 9A-9B are sets of representative images of the measurements of IL-6 in patient samples using the IL-6 VFA. As referred to herein, RA refers to rheumatoid arthritis samples and HC refers to healthy control samples. Twenty (20) RA (FIG. 9A) and 20 HC (FIG. 9B) serum samples were tested using the IL-6 VFA by following Assay Protocol III. FIGS. 9A and 9B are sets of representative images of the IL-6 VFA run using 20 healthy controls (FIG. 9B) and 20 RA (FIG. 9A) serum samples. All VFAs employed Amersham 0.2 μm with two layers. VFAs showed varying signals on the test zone (top left quadrant), a strong signal at Std-1 (top right quadrant), an intermediate signal at Std-2 (bottom left quadrant), and no signal at Std-3 (bottom right quadrant). Each subject's IL-6 level was accorded a semi-quantitative score, ranging from 0, 1+, 2+, 3+, 4+, and 5+; a semi-quantitative imaging score, ranging from 0, 0.5+, 1+, 2+, 3+, 4+, 5+.

FIGS. 10A-10E are graphical representations to demonstrate the discriminating power of IL-6 VFA and commercial ELISA. As referred to herein, RA refers to rheumatoid arthritis samples, HC refers to healthy control samples, IS refers to an imaging score, and OS refers to an observer score. FIGS. 10A-C are graphical representations of IL-6 levels in 20 RA serum and 20 HC serum as assayed using a commercial IL-6 ELISA, IL-6 VFA with IS readouts, and IL-6 VFA with OS readouts. IL-6 level group comparisons were analyzed using the Mann-Whitney U-test (** p<0.01). IL-6 levels in RA were significantly higher than in HC by all 3 readouts. FIG. 10D is a graphical representation of the correlation between VFA by IS and VFA by OS showed a Spearman correlation coefficient of 0.88, p<0.0001. FIG. 10E is a graphical representation of the correlation between IL-6 by ELISA and VFA by OS, as plotted with a Spearman correlation coefficient=0.56. p=0.0002.

FIGS. 11A-11C depict supplemental evaluation of buffer, membrane type, and membrane layer numbers on performance of 150 nm streptavidin GNP-based (150-SA-GNP) IL-6 VFA. FIG. 11A illustrates two layers of Cytiva NCM 0.2 μm or 0.45 μm that were assembled into the VFA. Each label displays the pore size and the number of layers of membrane tested. The top left quadrant is marked as “+” for a sample spiked with 100 ng/mL rIL-6 and the bottom right quadrant is marked as “−” for a non-spiked sample. The “+” (100 ng/mL) zone is expected to appear as a homogenous, uniform green spot with maximum intensity, while the “−” (0 pg/mL) test zone should show no signal. The 0.45 μm pore size NCM outperformed 0.2 μm NCM using Assay Protocol IV (a,b) and 0.2 μm pore size NCM outperformed 0.45 μm NCM using Assay Protocol V (c,d). FIG. 11B depicts, in the first row, three types of porous membranes (0.2 μm, 0.45 μm, and 5 μm) that were evaluated to detect 100 ng/mL rIL-6 (+) and non-spiked (−) samples using double-stacked layers of membrane (a-c). The second row depicts the same three types of porous membranes that were evaluated to detect 100 ng/mL rIL-6 (+) and non-spiked (−) samples using single-stacked layers of membrane (d-f). The 0.2 μm NCM showed stronger positive signal when using two layers of NCM, while 0.45 μm and 5 μm NCM showed a comparable positive signal with either one or two layers of NCM. FIG. 11C depicts, in the first row, images corresponding to IL-6 serial dilution, assayed using 0.2 μm NCM, following Assay Protocol II. The second row depicts images corresponding to IL-6 serial dilution, assayed using on 0.45 μm NCM using Assay Protocol I. Amersham 0.2 μm membrane showed higher sensitivity than Amersham 0.45 μm.

FIG. 12 depicts supplemental evaluation of characteristics of 40 nm streptavidin GNP-based (40-SA-GNP) VFA. Given the limited sensitivity of 150-SA-GNP, 40-SA-GNP was next evaluated. In total, 1 μL of 1 mg/mL anti-IL-6 was spotted on top left quadrant (+pg/mL of rIL-6) and lower right quadrant (0 pg/mL) on 0.2 μm NCM (Cytiva). Two (a), three (b), and four (c) layers of 0.2 μm NCM (Cytiva) were assembled with two layers of CF5 and one layer of CF7 absorbent pad. Following Assay Protocol V, rIL-6-spiked buffer-2 (200 pg/mL or 50 pg/mL) and non-spiked (0 ng/mL) samples were loaded to the top left quadrant and lower right quadrant, respectively. Position of test zone shifted dramatically when using three NCM layers, while a wide area of positive signal appeared when using four NCM layers, thus rendering two NCM layers an optimal choice for this particular embodiment. Following Assay Protocol V, two layers of 0.2 μm NCM (d), and two layers of 0.45 μm NCM (e) were assembled, and both showed a dim signal at the top left quadrant of NCM for detecting 10 pg/mL of rIL-6 and no background at the lower right quadrant of NCM for detecting non-spiked buffer-2 (0 pg/mL).

### DETAILED DESCRIPTION

Vertical flow assay devices, systems, and methods are described herein for detecting presence or an amount of cytokines in a sample. Provided herein are immunoassay methods for detecting presence or an amount of IL-6 in a sample, using a vertical flow assay device. The vertical flow assay devices, systems, and methods of the present disclosure can be used to measure any analyte, for example, cytokines, for example, IL1-alpha, IL1-beta, TNF-alpha, IL-2, IL-3, IL-4, IL-6, IL-7, IL-9, IL-10, IL-11, IL-12, IL-13, IL-16, IL-17, IL-18, LT, LIF, CXCL10/IP-10, CCL2/MPC-1, MIP-1beta, matrix metaloproteinase-9, oncostatin, or IFN-alpha, IFN-beta, IFN-gamma, TGF-beta, and G-CSF.

As used herein, the singular forms “a,” “an,” and “the” include plural referents unless the context clearly dictates otherwise. The term “about” is used to indicate a deviation of +/−2% of the given value, preferably +/−5%, and most preferably +/−10% of the numeric values, where applicable.

### IL-6 as Marker of Inflammation and Prognosis

IL-6 is a soluble mediator with a pleiotropic effect on inflammation, immune response, and hematopoiesis. IL-6 is part of a complex, interdepending network of cytokines released in inflammatory conditions.

IL-6 also plays a key role on T-cell activation and proliferation and B-cell differentiation. The neutralization of IL-6 with antibodies specific to the cytokine or the α-chain receptor improves inflammation in pre-clinical models. Additionally, during acute phase response, various proteins increase 10-100-fold, and IL-6 was found to be a key inducer of this response. It was also noted that IL-6 levels increase rapidly after infection, as seen upon injecting lipopolysaccharide in mice or following acute viral or bacterial infections in humans. Elevated IL-6 levels are present in several pathological states. For instance, IL-6 levels are significantly elevated in obese patients with uncontrolled type 2 diabetes mellitus and are even predictive of disease severity. IL-6 plays a role in chronic inflammatory conditions such as rheumatoid arthritis, inflammatory bowel disease, Castlemann's disease, hematopoietic diseases, and after physical stress such as surgery or chemotherapy. Increased levels of serum and/or tumor IL-6 are also seen in a number of malignant conditions, both hematopoietic malignancies and solid tumors including breast, cervical, esophageal, head-and-neck, ovarian, prostate, colorectal, pancreatic, hepatocellular, gall bladder, non-small-cell lung cancer and multiple myeloma, reflecting the immunological involvement in cancer. Several studies have shown IL-6 to be a prognostic indicator of survival as well as predictive in response to therapy in many types of cancer. A high IL-6 level is generally associated with a poorer outcome, particularly regarding renal cell, ovarian and prostate cancer, and correlated to more severe symptoms in regards to cancer as well as the development of anti-cancer drug resistance. Further, IL-6 can be a prognostic marker and a predictor of outcome of COVID-19 infection. An amount of IL-6 as low as 10 pg/mL to as high as 10,000 pg/mL or more in biological samples can be relevant for diagnosis, prognosis, monitoring, and treatment selection purposes. Other diseases with elevated IL-6 levels are listed in Table 1.

TABLE 1
The mean levels of IL-6 reported in blood, serum, or plasma
of healthy controls and various inflammatory diseases.

| Condition | Mean IL-6 levels (range) | |
|---|---|---|
| Healthy control | 5.2 | pg/mL (0-7) |
| COVID-19 induced CRS† | 40 | pg/mL (34.5-214.5) |
| Irritable bowel syndrome | 1.1 | pg/ml (0.5-1.6) |
| Obstructive Sleep Apnea | 1.2 | pg/ml (1.1-1.4) |
| Fibromyalgia Syndrome | 134.9 | pg/mL (67.6-202.2) |
| Rheumatoid Arthritis | 35.1 | pg/ml (13.8-56.4) |
| Atrial Fibrillation | 14 | pg/mL (0.2-156.5) |
| Atypical Depression | 2.5 | pg/mL (1.2-3.8) |
| Breast Cancer | 111.4 | pg/mL |
| Sepsis | 3.5 | ng/mL (<0.1-305) |
| COVID-19 Pneumonia | 1000 | pg/mL (20-10,000) |
| CAR-T cell therapy induced | 3000 | pg/ml |
| Cytokine release syndrome | | |
| Lung cancer (Stage 4) | 36.5 | pg/mL (20-400) |
| Pneumonia | 2852 | pg/ml |
| Polycystic ovary syndrome | 4.8 | pg/mL (0-1000) |
| Ovarian cancer | 55.6 | pg/mL (0-2869) |
| Renal cell carcinoma | 224 | pg/mL (62-707) |
| Pancreatic cancer | 3-156 | pg/ml |

### Components and Assembly Of Vertical Flow Assay

An embodiment includes a vertical flow assay device for detecting presence or an amount of a cytokine in a sample. One such device contains (a) a membrane member having a first surface and a second surface, (b) a capture agent capable of specific binding to a cytokine and immobilized on or within the membrane member, (c) an absorbent pad member having a first surface and a second surface, and located under the membrane member, wherein the second surface of the membrane member and the first surface of the absorbent pad member are in contact with each other, (d) a holder with a first wall and a second wall each having a first surface and a second surface. The first wall of the holder is positioned proximal to the membrane member, such that the second surface of the first wall and the first surface of the membrane member are proximal to each other. This first wall has a cavity that extends from the first surface to the second surface of the first wall of the holder. This cavity exposes a portion of the first surface of the membrane member. The second wall is proximal to the absorbent pad member, such that the second surface of the absorbent pad member and the first surface of the second wall are proximal to each other. The holder is a tight-fit configuration that provides a chamber to contain the membrane member and the absorbent pad member.

In some embodiments, the membrane member of the vertical flow assay device is made of one or more porous solid state materials. These materials can be plastic, polymers, paper, nitrocellulose, cellulose, PVDF, polycarbonate, ceramic, metallic materials, glass, glass microfibers, and anodized aluminum. In some embodiments, the membrane member is a nitrocellulose membrane. In some embodiments, the membrane member has pores that are 10 micrometers (μm) or less in size and a porosity of between 10% and 95%. In certain embodiments, the membrane member has pores that are about 0.1 μm to about 0.5 μm in size. Certain membrane members can have pores sizes of about 0.2 μm or about 0.45 μm.

In some embodiments, the absorbent pad member of the vertical flow assay device is made of one or more absorbent pads. In some embodiments, the absorbent pad has pores that are between 1 μm and 1000 m in size, and can be spatially arranged to provide a substantially uniform flow rate profile across the membrane member. In certain embodiments, the absorbent pad has thickness of between 0.1 mm to 10 mm at 53 kilopascal (kPa). The absorbent pads can be made of cellulose, mixed cellulose ester, cotton, activated carbon, or glass fibers. Certain embodiments of the absorbent pads include one or more of Whatman® CF3 (322 μm thickness at 53 kPA), CF4 (482 μm thickness at 53 kPA), CF5 (954 μm thickness at 53 kPA), CF7 (1873 μm thickness at 53 kPA), and GR470 (840 μm thickness at 53 kPA). The absorbent pads have a wicking rate ranging from about 30 to about 200 s/4 cm and can quickly absorb the biological sample with a water absorption rate of about 30 to about 300 mg/cm².

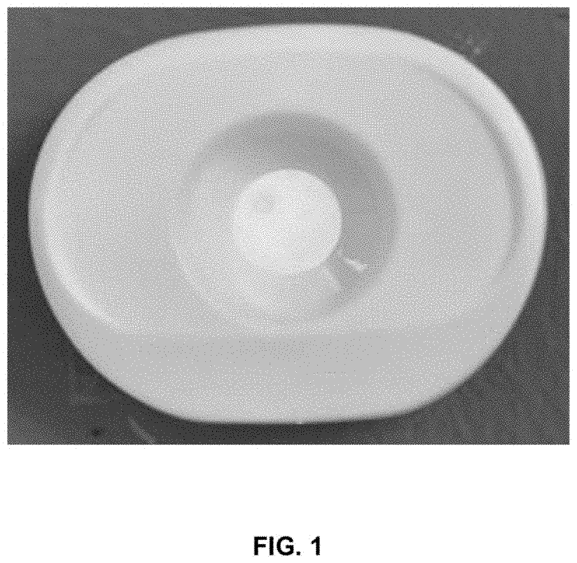

A top view of a vertical flow assay device is shown in FIG. 1. This view shows the holder with an opening that traverses the first wall of the holder to expose a portion of the first surface of the membrane member.

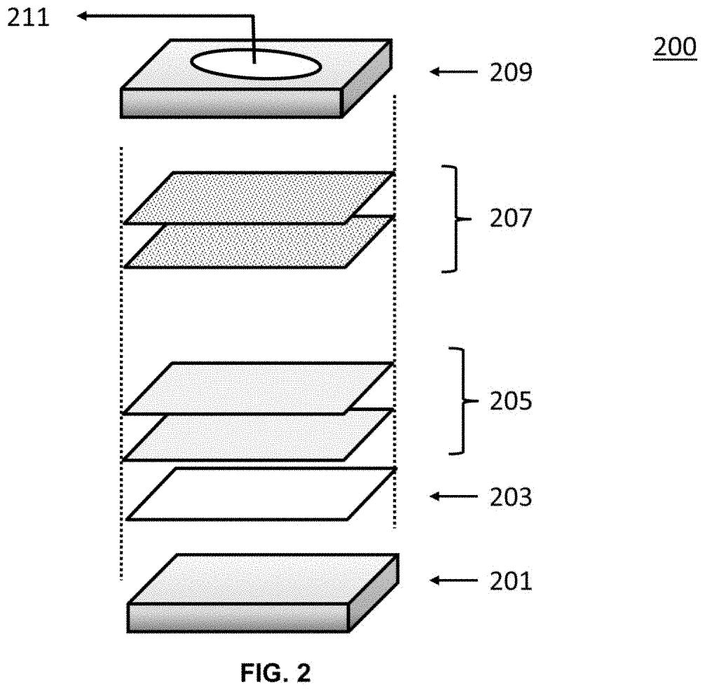

The components and assembly of the device is schematically shown in FIG. 2. The holder, such as an external plastic housing or a “cartridge”, comprises a second wall [201] and a first wall [209]. The membrane member [207] can include one or more porous solid state materials. The membrane member can be made from nitrocellulose, but numerous other materials for physical absorption or covalent coupling are well known to the person skilled in the art. Examples of membranes include, but are not limited to nitrocellulose membranes, blotting membranes, diethylaminoethyl ion exchange paper and blot adsorbent filter papers. The membrane member can be made of plastic, polymers, paper, nitrocellulose, cellulose, PVDF, polycarbonate, ceramic, metallic materials, glass, glass microfibers, and anodized aluminum. In some specific embodiments, the membrane member is one or more nitrocellulose membrane.

The absorbent pad member [205] can include one or more absorbent pads of varying thicknesses. The absorbent pads can be made from cotton, for example, a 100% cotton linter material. Any support material can be used as absorbent pads. In certain embodiments, it has functional groups to which an adsorbent molecule (either a receptor or a ligand) can be bound. Examples of such materials include aminobenzyl-oxymethyl (ABM) paper, 2-aminophenyl thioether (APT) paper, cyanogen bromide activated paper (CBA) (see Methods in Enzymology, R. Wu (ed.) 1979, Academic Press New York, 68:436-442 for a discussion of CBA paper), diazobenzyloxymethyl cellulose paper (DBM), diazophenylthioether cellulose paper (DPT) and nitrobenzyloxymethyl cellulose paper (NBM). In some embodiments, the vertical flow assay device can contain one or more absorbent pad members that include one or more absorbent pads. In some embodiments, the absorbent pad member comprises pores that are between 1 μm and 1000 m in size, and are spatially arranged to provide a substantially uniform flow rate profile across the membrane member; and wherein the absorbent pad member has thickness of between 1 mm to 10 mm at 53 kPa. In some embodiments, the absorbent pad member has thickness of about 4.7 mm at 53 kPa.

According to an aspect of both the device and the method, freely combinable with the above mentioned aspects, the membrane member has a detection surface, onto which the sample and reagents is/are applied. The area of the detection surface can be smaller than the total area of the membrane member and/or the absorbent pad member positioned under the membrane member. The detection surface can be about the same size as the opening in the first wall of the holder. The ratio of area of the detection surface of the membrane member to the area of the absorbent pad member can be about 1:2, about 1:4, or about 1:8. This ratio can also be expressed as the capacity of the absorption pad in relation to the total volume of sample and reagents to be added. In certain embodiments, the absorption pad has the capacity to absorb more than 100% of the total volume of sample and reagents to be added, more than 150%, or more than 200% or more of the total volume of sample and reagents to be added.

The vertical flow assay cartridge (or a device) [200] can be assembled by positioning layers of absorbent pads [203, 205] on the second wall [201] of the plastic housing. The membrane member [207] is then positioned onto the stacked absorbent pads [203, 205]. The first wall [209] of the plastic housing can be then positioned above the NCM [207]. The second wall [201] and the first wall [209] of the plastic housing can provide a tight configuration to provide a chamber, such as tight-fit chamber. The first wall [209] of the plastic housing can be pressed down to the second wall [201] to attach the first wall [209] to the second wall [201] and to close the cartridge. The first wall [209] comprises an opening [211] (“a test window”) which allows contacting the material thereunder, in this example the NCM [207], from outside the cartridge when the cartridge is closed.

In some embodiments, the holder includes a standard fitting configured to mount to a detection system. A portion of the membrane member can be accessed through the opening of the first wall of the holder to provide a detectable signal indicative of the presence and/or the amount of the cytokine to the detection system. In some embodiments, the detection system includes an imager, a scanner, or a smartphone.

In some embodiments, the vertical flow assay device provides the minimal detectable concentration of IL-6 in the sample, for example, a serum sample is about 3.2 pg/ml or less. In some embodiments the vertical flow assay device provides the reporting range of IL-6 from about 10 pg/ml or less to about 1,000 pg/ml or more in a sample, for example, a serum sample. In some embodiments, the vertical flow assay device further comprises a positive control spot and/or a negative control spot on the membrane member. In some embodiments, the vertical flow assay device is disposable and/or for single use.

Disclosed herein are kits for the analysis of a cytokine. The kit can include reagents for the analysis of at least one test sample, which comprises at least one antibody that specifically binds to the cytokine. The kit can also include devices and instructions for performing one or more of the diagnostic and/or prognostic correlations described herein. Embodiments of kits can include an antibody pair for performing a sandwich assay, or a labeled species for performing a competitive assay, for the analyte. In certain embodiments, an antibody pair comprises a first (capture) antibody conjugated to a solid phase, a second (detection) antibody, and a detectable label or a signal development element bound or bindable to the second (detection) antibody, wherein each of the first and second antibodies bind to the cytokine. In certain embodiments, each of the antibodies are monoclonal antibodies. The instructions for use of the kit and performing the correlations can be in the form of labeling, which refers to any written or recorded material that is attached to, or otherwise accompanies a kit at any time during its manufacture, transport, sale, or use. For example, the term “labeling” encompasses advertising leaflets and brochures, packaging materials, instructions, links to electronic materials, as well as writing imprinted directly on kits. The kit may also contain a washing buffer and/or a blocking buffer. Any washing buffer and/or blocking buffer for conducting immunoassay can be used. For example, washing buffer can be phosphate-buffered saline optionally comprising Tween-20. Blocking buffer can be washing buffer comprising an amount of a blocking agent, for example, fetal bovine serum or bovine serum albumin. The kit can be an in vitro diagnostics. The term “in vitro diagnostic” as used herein refers to a medical device which is a reagent, reagent product, calibrator, control material, kit, instrument, apparatus, equipment, or system, whether used alone or in combination, intended by the manufacturer to be used in vitro for the examination of specimens, including blood and tissue samples, derived from the human body. These samples are analyzed for the purpose of providing information concerning a physiological or pathological state, or concerning an abnormality, or to determine the safety and efficacy of certain treatment regimens, or to monitor therapeutic measures. Certain embodiments include a kit for detecting presence or an amount of a cytokine, for example, interleukin 6 (IL-6) in a sample. This kit includes one or more of a vertical flow assay device of the present disclosure, a capture reagent and a detection reagent that each bind to a cytokine, such as IL-6, a signal development element that can bind to the detection reagent, a washing buffer, a blocking buffer, a calibration marker to relate a signal to the presence or the amount of a cytokine, such as IL-6; and/or an instruction for use.

### Detecting Cytokine Levels in Samples

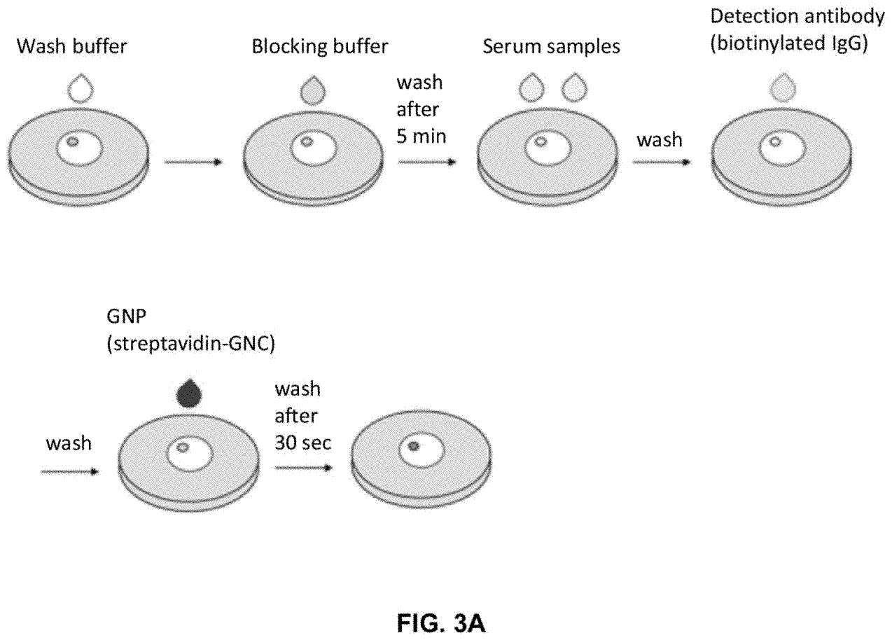

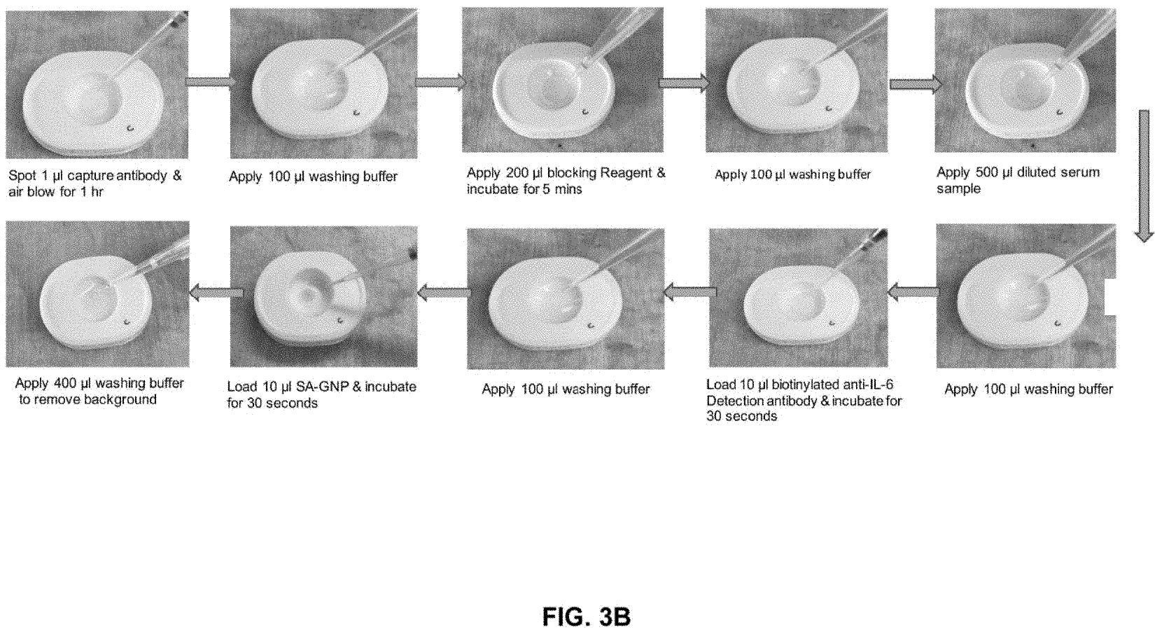

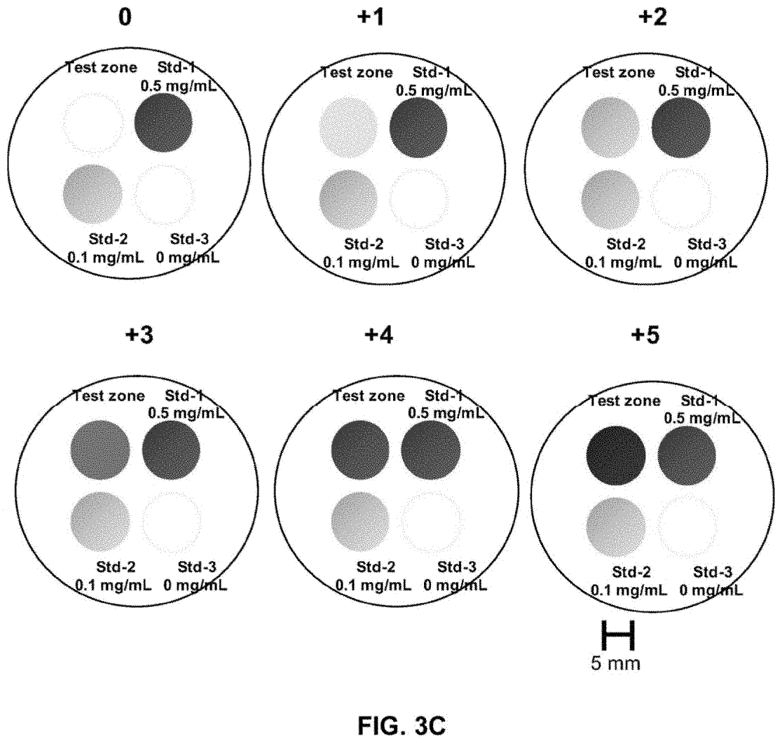

Using the vertical flow assay cartridge, cytokine levels in samples can be measured as schematically depicted in FIG. 3A. First, an immunomembrane can be prepared by immobilizing an anti-IL-6 capture antibody on the membrane member [207], for example, any one of the four corners of the membrane member, such that the antibody was immobilized in the test zone [211]. The term “immunomembrane” refers to a porous membrane which allows liquids and very small particles suspended in a liquid to pass through the membrane, and which has antibodies immobilized to the solid material of the membrane. In an embodiment, an immunomembrane is made from nitrocellulose or another membrane suitable for immobilizing antibodies or other binding molecules or fragments thereof. The membrane can have an average pore size of less than 10 μm, and in certain embodiments about 0.2 μm. The vertical flow assay device containing the immunomembrane can be stored at room temperature, for example, in desiccator pouches, until needed, for example, for 30 days, 60 days, or 90 days. Other examples of assay use and interpretation are provided with reference to FIGS. 3B and 3C, which are described below.

Vertical flow assay for detecting presence or an amount of a cytokine in a sample can be run as follows:
applying a sample onto the membrane member through the opening of the first wall of the holder onto the test zone [211];
applying a detection agent that binds to a cytokine, thereby forming a sandwich complex comprising the capture agent, the cytokine, and the detection agent;
providing a detectable signal that is related to the presence or amount of the cytokine in the sample bound in the sandwich complex; and
relating the detectable signal to the presence or the amount of the cytokine in the sample.

In some embodiments, the capture agent and/or the detection agent is an antibody that binds to a cytokine, such as IL-6. In some embodiments, the detection agent is bound to a detectable label or a signal development element. Detectable labels or signal development elements may include molecules that are themselves detectable (for example, fluorescent moieties, electrochemical labels, and/or metal chelates) as well as molecules that may be indirectly detected by production of a detectable reaction product (for example, enzymes such as horseradish peroxidase or alkaline phosphatase) or by a specific binding molecule which itself may be detectable (for example, biotin, digoxigenin, maltose, oligohistidine, 2,4-dintrobenzene, phenylarsenate, ssDNA, and/or dsDNA). Detectable labels and signal development elements can comprise any other signal systems that provide signals indicative of the cytokine levels in the sample bound in the sandwich complex. In some embodiments, the signal development element comprises gold nanoparticles. Gold nanoparticles can be about 1-500 nm in diameter, for example, about 1, 5, 10, 20, 30, 40, 50, 60, 70, 80, 90, 100, 200, 300, 400, 500 nm in diameter. In some embodiments, the gold nanoparticles are about 40 nm in diameter. Gold nanoparticles can be conjugated to streptavidin, and can bind to a biotin-conjugated detection agent.

The assay may produce a visual signal, such as color change, fluorescence, luminescence, and the like, when indicating the presence or absence of an analyte in a sample. There is a direct relationship between the amount of analyte (for example, IL-6) molecules and the signal (for example, color) to be measured, since the amount of signal particles (for example, gold nanoparticles) bound relates to the amount of analyte molecules present in the sample to be tested. This signal is then detectable either visually with comparison to pre-evaluated, pre-calibrated and/or predetermined signal diagrams or by measurement of the amount of signal by electronic signal detectors either freely available on the marked or a specific device developed for the disclosed methods and devices.

In the vertical flow assay methods of the present disclosure, at least one reference spot of colored material can be arranged on the assay. In some embodiments, the vertical flow assay device further comprises a positive control spot and/or a negative control spot on the membrane member. Positive controls can comprise a cytokine or fragments thereof bound to the immobilized capture agent. Negative controls can comprise the absence of a capture agent.

In an embodiment, one or more reference spots (for example, positive controls, negative controls) are positioned or fastened in close proximity to the membrane with immobilized antibodies or bother binding molecules or fragments thereof, such as on the holder of the assay membrane. When measuring the signal (for example, reflectance) from the membrane, this or these reference spots are measured as well. The reading obtained at said at least one reference spot can be used to compensate for instrument-to-instrument and other hardware variations, and to increase the overall accuracy of the assay.

A reference value can be obtained by measuring the intensity of the signals of a reference spot arranged on the assay. The reference spot can be manufactured separately or integrally with the assay device, using a known concentration of the same label used on the labeled particles. Techniques for providing a reference spot on an immunometric assay are known to persons skilled in the art.

In some embodiments, the immunoassay method further comprises mounting the vertical flow assay device to a detection system to provide the signal, and determining the presence or amount of a cytokine, such as IL-6, in the sample. In some embodiments, the detection system includes an imager, a scanner, or a smartphone. The detection can be performed easily and quickly, for example, without dissembling the vertical immunoassay device, in point-of-care settings, for example, at a clinic or at home. The result, such as the signal intensity, can be read or measured within several minutes after the test having been performed. Drying of the test device and the membranes composed—and the microfluidic flow caused by storage and by drying—may however change the signal intensity on the test surface. The stability of signal particles (for example, gold nanoparticles) bound to IL-6 in the immunomembrane is sensitive, dependent on blood reflux into the detection membrane, temperature, humidity and/or time elapsed since application of any of the three reagents. This time frame is adequate for user to be able to manually and/or electronically evaluate or measure the color intensity.

An analyte is measured in a sample. Such a sample may be obtained from a subject, or may be obtained from biological materials intended to be provided to the subject. The term “subject” as used herein refers to a human or non-human organism. While a subject is a living organism in most instances, the methods and devices described herein may be used in post-mortem analysis as well. “Patients” as used herein refer to living humans that are receiving medical care or medical examination for a disease or condition. For example, a sample may be obtained from a cancer patient, a sepsis patient, a patient having infection, for example, COVID-19 infection, a patient experiencing cytokine release syndrome (CRS), for example, CAR-T therapy induced CRS, a patient having inflammatory disease, for example, rheumatoid arthritis.

The term “body fluid sample” as used herein refers to a sample of bodily fluid obtained for the purpose of diagnosis, prognosis, classification, or evaluation of a subject of interest, such as a patient or transplant donor. In certain embodiments, such a sample may be obtained for the purpose of determining the outcome of an ongoing condition or the effect of a treatment regimen on a condition. Embodiments of body fluid samples include blood, serum, plasma, cerebrospinal fluid, urine, saliva, sputum, and pleural effusions. In addition, one of skill in the art would realize that certain body fluid samples would be more readily analyzed following a fractionation or purification procedure, for example, separation of whole blood into serum or plasma components. A sample can be whole blood, blood plasma, blood serum, and blood hemolysate. In some embodiments, a sample is a serum sample obtained from a patient. Additionally or alternatively, a sample can be taken from an animal, for example but not limited to humans, domesticated animals such as pets and utility animals, such as dogs, cats, horses, camels, donkeys, and birds, and livestock, such as cattle, pigs, sheep, and goats.

The vertical flow assay devices and methods provided herein can detect a wide range of concentrations of a cytokine in a sample. For example, the vertical flow assay devices and methods of the present disclosure can detect a cytokine in a sample with a minimum detection concentration of 1000 pg/mL or less, 900 pg/mL or less, 800 pg/mL or less, 700 pg/mL or less, 600 pg/mL or less, 500 pg/mL or less, 400 pg/mL or less, 300 pg/mL or less, 200 pg/mL or less, 100 pg/mL or less, 50 pg/mL or less, 10 pg/mL or less, 5 pg/mL or less, 4 pg/mL or less, 3 pg/mL or less, 2 pg/mL or less, or 1 pg/mL or less. The vertical flow assay devices and methods of the present disclosure can detect a cytokine in a sample with a detection range of 100-5,000 pg/mL, 100-10,000 pg/mL, 50-5,000 pg/mL, 50-10,000 pg/mL, 10-5,000 pg/mL, 10-10,000 pg/mL, 5-5,000 pg/mL, 5-10,000 pg/mL, 4-5,000 pg/mL, 4-10,000 pg/mL, 3-5,000 pg/mL, 3-10,000 pg/mL, 2-5,000 pg/mL, 2-10,000 pg/mL, 1-5,000 pg/mL, or 1-10,000 pg/mL. In some specific embodiments, the vertical flow assay devices and methods of the present disclosure can detect IL-6 in serum samples in the range of 10-10,000 pg/mL.

The vertical flow assay devices and methods provided herein can provide results of the presence or amount of a cytokine in a sample in a rapid manner. For example, the vertical flow assay devices and methods provided herein can provide results of the presence or amount of a cytokine in a sample within 240 minutes, 180 minutes, 120 minutes, 60 minutes, 50 minutes, 40 minutes, 30 minutes, 25 minutes, 20 minutes, 15 minutes, 10 minutes, or less from the time the sample is applied to the vertical flow assay device of the present disclosure.

The methods and systems of the present disclosure for detecting a cytokine offers advantages over existing methods and systems. A common existing method for detecting a cytokine is enzyme linked immunosorbent assay (ELISA), wherein a varying range of a cytokine levels can be detected. ELISA requires two-days for the completion, and is hence not suitable for use as a rapid test for point-of-care applications, for example, routine monitoring at clinic or at home.

Another existing method for detecting a cytokine is a Lateral Flow Assay (LFA). In conducting a sandwich immunofluorescent lateral flow assay to detect IL-6 over two hundred serum samples, the lower detection limit for LFAs was found to be 0.38 ng/mL, with a linear range between 1.25 ng/mL and 9,000 ng/mL for IL-6. It took approximately 20 minutes to obtain results. The low sensitivity (0.38 ng/mL) is a major disadvantage. In diagnosis or monitoring of certain diseases or conditions, for example, cytokine release syndrome (CRS) in cancer patients under chimeric antigen receptor T cells (CAR-T) therapy or patients with COVID-19 infection, IL-6 levels as low as 10-100 pg/mL need to be detected.

The vertical flow assay (VFA) systems and methods of the present disclosure overcome these limitations in the existing systems and methods. Embodiments of methods and systems for cytokine detection provided herein are rapid, have high multiplex capability, high sensitivity, and larger volume capacity. Further, the devices according to the present disclosure are inexpensive to create and can be used in low-resource settings, such as homes, for diagnosis, requiring no special equipment.

The present disclosure provides a new platform technology for a semi-quantitative point-of-care vertical-flow assays, to detect a cytokine within 15 min. For example, the minimum detectable concentration of IL-6 can be about 3.2 pg/mL in a serum sample. The reportable range of IL-6 can be from about 10 pg/ml or less to about 1,000 pg/ml or more in a serum sample. The use of inexpensive, stable, colorimetric gold nanoparticles as VFA reporters offers substantial advantages of sensitivity and quantifiability. The systems and methods of the present disclosure meet the unmet need for clinically-actionable point-of-care and/or home monitoring of inflammation disease tracking and introduce a new general platform technology. Following simple steps, signal intensity shows the potential to track with disease activity serially. Further, the VFA devices with immobilized cytokine capture antibodies can have at least three-month stability at room temperature storage.

The methods provided herein are fast (approximately 15 minutes from start to completion), and makes it possible to perform the analysis and reach a result of diagnostic value, in the clinic, at the bedside, in the home, or in the field. The test is also simple and cheap to manufacture, and it can be used without the need for expensive, auxiliary equipment, such as electronic readers or other similar equipment.

The relationship between the area of the detection zone and the area or rather absorption capacity of the absorbing pad offers a particular advantage in that all the sample and liquid reagents are drawn through the membrane, leaving only the antibody-analyte sandwich complex bound to the membrane. This results in an improved signal versus background ratio, and it reduces or eliminates problems with back-flow of sample or reagents, which makes it possible to read the result of the assay within a prolonged period, for example up to 30 minutes after performing the assay. This in turn makes it possible to perform several assays at the same time.

In an embodiment, the VFA system includes plastic housing sourced from Cytodiagnostics. In contrast to the commercially available VFA cartridge that only contains one layer of membrane and one layer of absorbent pad, an embodiment of the VFA system contains two layers of optimized Amersham 0.2 μm pore-sized membrane and multiple layers of the absorbent pad. This embodiment of the VFA system has a wide range of benefits, which include, but are not limited to, excellent signal-to-noise ratios without the need for rigorous washing conditions, reduced “blow-through” risk, high binding capacity of small proteins, longer shelf life of proteins on the transfer membrane, and excellent sensitivity, resolution, and low background. By using multiple layers of both the membrane and absorbent pad, this embodiment of the VFA system ensures a strong, homogenous signal in the test zones and proper absorption of any buffers or other media that pass through the membrane.

The commercially available VFA requires that gold nanoparticles with an OD value of 10 be used to visualize the signal in the test zone. This embodiment of the VFA system can sustain OD values as low as OD=2, thereby conserving the amount of gold nanoparticles used. Moreover, the VFA has incredibly high multiplex capability that is not affected by the amount of body fluid volume added due to the optimization of the absorbent pads. The commercially available VFA requires that the volume spotted be significantly reduced to achieve the same multiplex capability.

### EXAMPLES

Various examples are described to illustrate selected aspects of the various embodiments of vertical flow assay devices, systems, and methods.

### Example 1: Evaluation of the Porous Membrane and Number of Layers

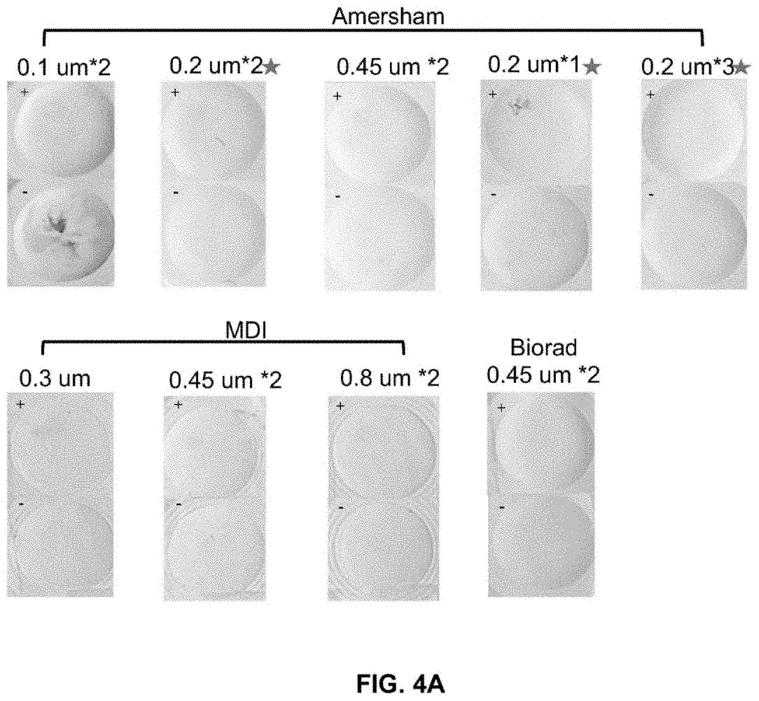

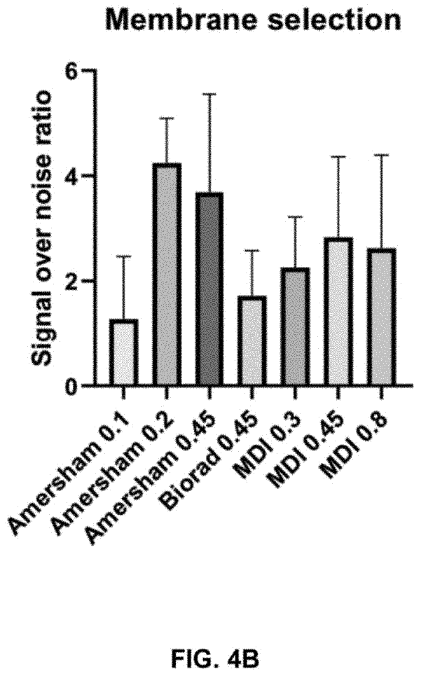

A sandwich immunoassay was adopted for the vertical flow paper-based assay. In this example, monoclonal anti-IL-6 antibody and biotinylated polyclonal anti-IL-6 antibody are used for targeting IL-6. Streptavidin-GNP is used to bind biotin on the detection antibody, to yield a red signal. Following Assay Protocol I (see Methods), seven types of NCMs were evaluated by comparing the signal over noise ratio (SNR) when assaying 10-fold diluted serum spiked with the analyte (100 pg/ml) or non-spiked (0 pg/mL). In this example, triplicates of each type of membrane were evaluated. FIG. 4A shows the quantification of SNR using seven types of NCM, while FIG. 4B summarizes the SNRs using the different types of NCM. The Amersham membrane with a pore size of 0.2 μm was selected as it had a relatively higher SNR and better morphology of spots. After selecting the make of the NCM for this assay, the number of layers of Amersham 0.2 μm membrane to use in the vertical flow stack was evaluated. As mentioned above, one, two, and three layers of Amersham 0.2 μm membrane were assembled with one layer of CF7 pad and two layers of CF5 pads, individually. Whatman® CF7 pad is a thick, 100% cotton linter material (1873 μm thickness at 53 kPA). Whatman® CF5 pad is a medium to thick weight, 100% cotton linter material (954 μm thickness at 53 kPA). The NCMs were spotted with capture antibody and tested using spiked and non-spiked serum in triplicate. Results from this example indicate that the two-layer set-up is suitable for serum IL-6 detection (FIG. 4A, NCMs with red asterisks).

In FIG. 4A, the two rows depict the seven types of porous membranes that were evaluated for detection of 100 pg/mL rIL-6 in 10-fold-diluted serum and non-spiked 10-fold diluted serum control. Triplicates were analyzed. Labels above each image provides information regarding the manufacturer from which the membrane was sourced, the pore size, and the number of layers of membrane tested. The test zone is at the top left corner of each set-up and is marked as +(spiked with 100 ng/ml IL-6) or as − (non-spiked sample). The +(100 pg/mL) test zone should appear as a homogenous, uniform pink spot with maximum intensity while the − (0 ng/mL) test zone should show no signal. The Amersham 0.2 μm membrane had the best intensity for the +control (100 pg/mL) and least background for the − control (0 ng/mL). It was selected and tested further using multiple stacked layers of that membrane as shown by the second, fourth, and fifth pictures, from the left, in the first row (all marked by red asterisks) which depict the single, double, and triple-stacked layers of Amersham 0.2 μm membrane. One of the more optimal membrane set-ups contains two layers of the Amersham 0.2 μm (Amersham 0.2 μm*2) as it displayed one of the strongest, homogenous signal in the positive test zone. FIG. 4B is a graphical representation of the colorimetric intensity values as calculated using Image J and the signal over noise ratio (SNR) was computed for each type of membrane. Amersham 0.45 μm and 0.2 μm also show relatively higher SNR. The error bar shows that the Amersham 0.2 μm membrane exhibits less variability. The error bar indicates the standard deviation of three measurements of SNR.

### Example 2: Dose Response Curve for the Vertical Flow Assay to Detect IL-6 in Serum

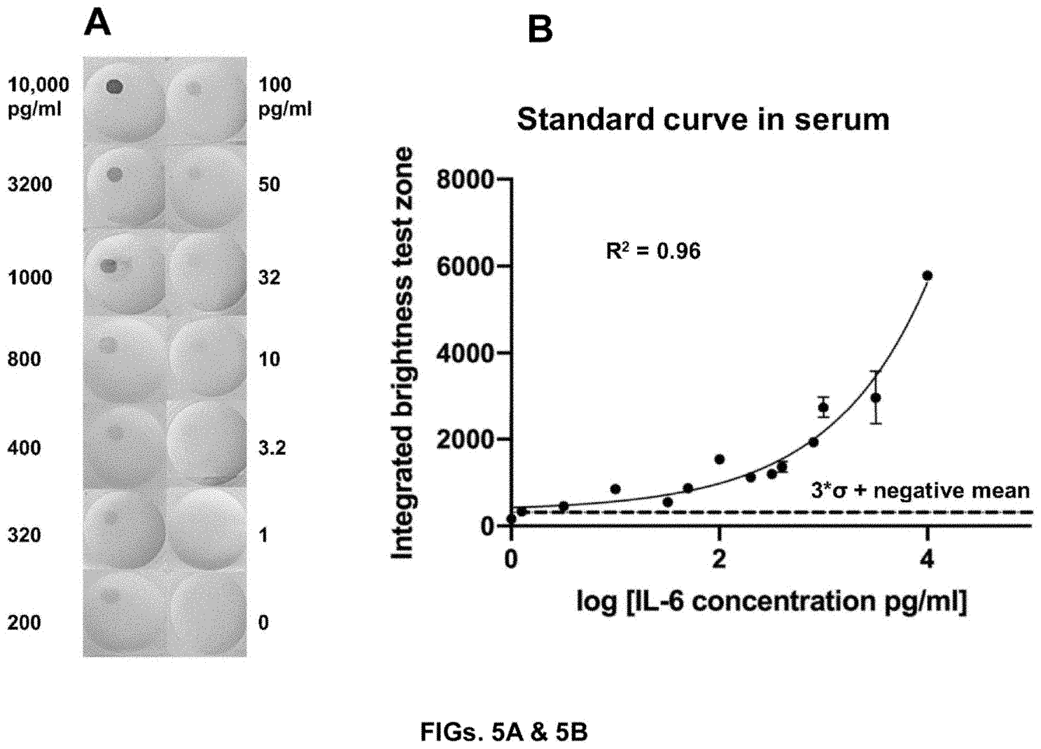

After evaluating the VFA structure and reagent concentration, the limit of detection of IL-6 in spiked serum was assessed. Following Assay Protocol II, ten-fold diluted healthy control serum was spiked with serial dilutions of IL-6. FIGS. 5A-5B depict a dose response curve for the vertical flow paper-based assays with IL-6 spiked into human healthy serum using the vertical-flow paper-based assay. FIG. 5A is a set of representative images of the assay results using two layers of Amersham 0.2 μm membrane. Two replicates were obtained at each concentration and three replicates for the negative. The top left of the membrane represents the test zone. As demonstrated in FIG. 5A, higher concentrations of IL-6 produced brighter red color at the top left corner. The color intensity of each image was analyzed using ImageJ and the limit of detection (LoD) was identified as the concentration greater than the mean intensity of the negative samples plus three standard deviations above the mean. FIG. 5B is a graphical representation of the colorimetric intensity values calculated using ImageJ. The 3*σ+negative mean line indicates the summation of mean intensity of non-spiked healthy serum samples (0 pg/ml) and 3× their standard deviation (σ). The concentration above this line marks the limit of detection, which is 3.2 pg/ml. The dose response curve demonstrated a reportable range from 3.2 pg/ml to 10,000 pg/ml of IL-6 in ten-fold diluted serum. Each point concentration was run in duplicates.

### Example 3: Reproducibility of the IL-6 Vertical Flow Assay

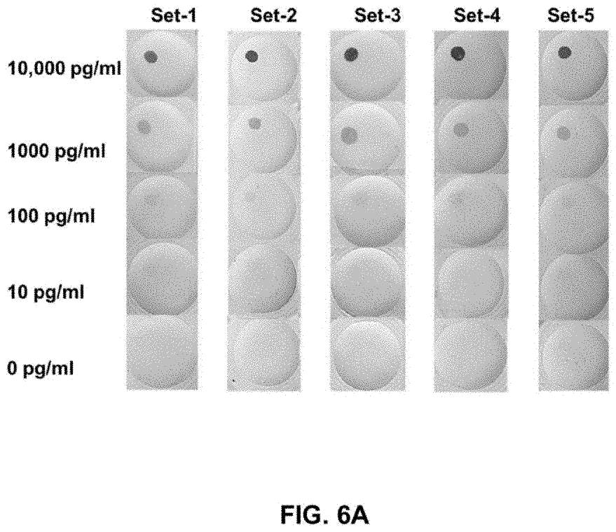

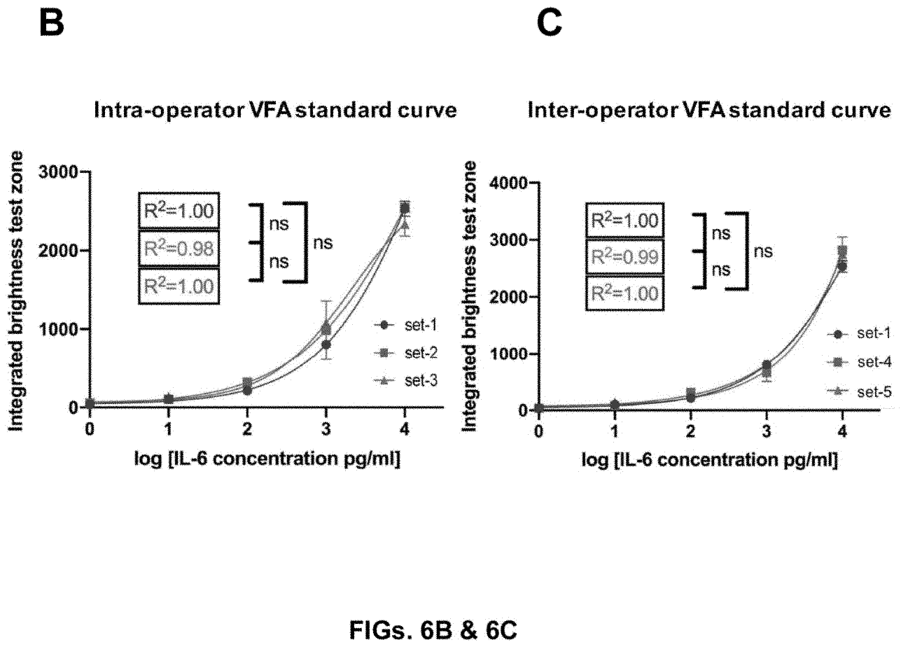

Following Assay Protocol II, the inter-operator coefficient variance (CV) was examined using assays performed by three researchers, and the intra-operator CV was examined using assays performed by one researcher, in all cases by testing standard curves of recombinant IL-6 spiked in buffer. Three researchers first ran three serial dilutions of IL-6 on the VFA (sets 1, 4, and 5 in FIG. 6A). The intra-operator CV was calculated as shown in Table 2 and FIG. 6B. Next, one researcher ran two additional serial dilutions of IL-6 on the VFA (sets 1, 2, and 3 in FIG. 6A). The inter-operator CV was calculated as shown in Table 3 and FIG. 6C. The CV comparison at each point concentration and the average CV for all experiments are shown in Tables 2 and 3, respectively. The relatively low inter and intra-operator CV values indicate the assay results are reproducible across multiple researchers and also when repeatedly performed by a single researcher.

TABLE 2
Inter-operator VFA performance

| IL-6 pg/ml | Set-1 | Set-2 | Set-3 | stdev | average | CV% |
|---|---|---|---|---|---|---|
| 10,000 | 2533.393 | 2817.32 | 2748.639 | 148.8085 | 2699.363 | 5.512723 |
| 1000 | 806.2938 | 663.9343 | 799.7019 | 80.3554 | 756.6428 | 10.61999 |
| 100 | 214.0874 | 316.0143 | 281.056 | 51.79442 | 270.3859 | 19.15574 |
| 10 | 165.5698 | 84.1768 | 146.4665 | 31.64964 | 112.071 | 28.24071 |
| 0 | 36.70013 | 39.6345 | 33.81197 | 2.011297 | 36.71553 | 7.920334 |

Average CV = 14.3

TABLE 3
Intra-operator VFA performance

| IL-6 pg/ml | Set-1 | Set-2 | Set-3 | stdev | average | CV% |
|---|---|---|---|---|---|---|
| 10,000 | 2533.393 | 2548.387 | 2338.512 | 119.9293 | 2472.363 | 4.7699 |
| 1000 | 806.2938 | 986.7507 | 1077.763 | 138.1698 | 956.9352 | 14.43878 |
| 100 | 214.0874 | 326.5484 | 268.2008 | 53.23346 | 267.6421 | 19.88979 |
| 10 | 105.5698 | 103.3036 | 135.0646 | 17.71929 | 114.646 | 15.45566 |
| 0 | 36.70013 | 51.9678 | 36.23533 | 8.951985 | 41.63342 | 21.5014 |

Average CV = 15.2

### Example 4: Stability of the IL-6 Vertical Flow Assay

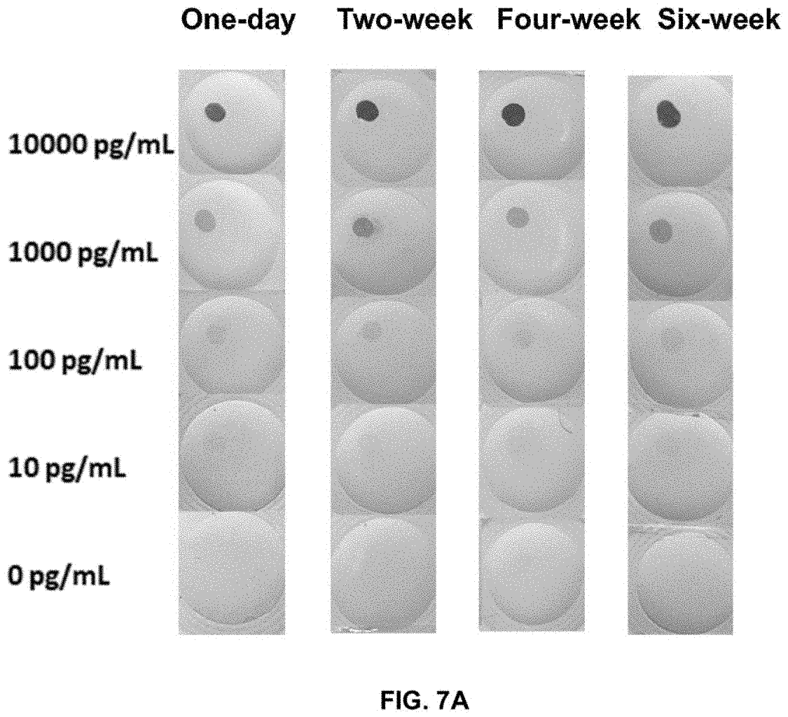

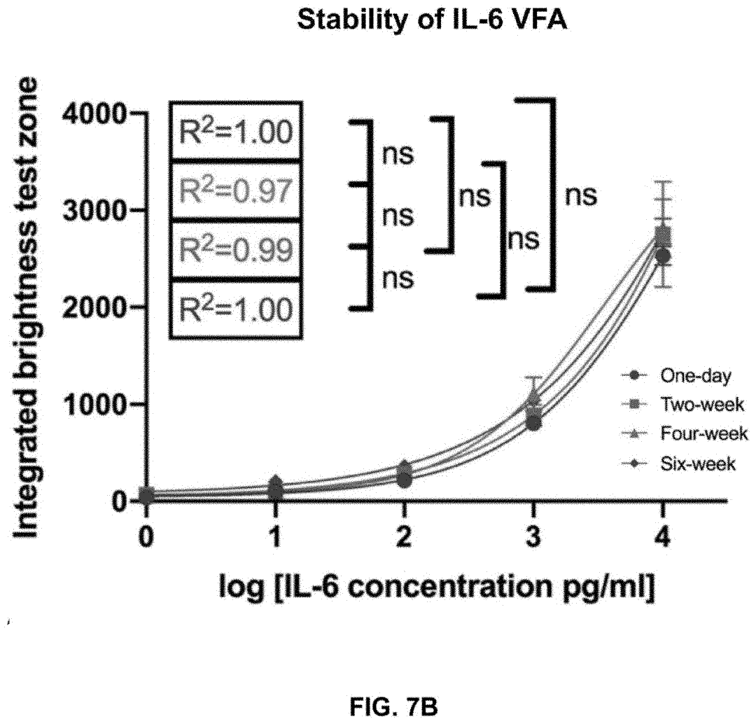

To examine the room temperature storage stability on VFA performance, VFA assay kits generated as described above were stored in desiccator bags at room temperature, marked as one day, two-week, four-week, and six-week, referring to the duration they were kept at room temperature. A serial dilution of IL-6 was tested on the stored VFA test kits, stored for varying lengths of time, as indicated. The images in FIG. 7A and quantification data in FIG. 7B demonstrate the impact of storage at room temperature. All four linear regression plots showed similar LOD, linearity and slope, indicating that the IL-6 VFA assay maintains its performance characteristics for at least 6 weeks at room temperature.

### Example 5: Specificity of rIL-6 VFA

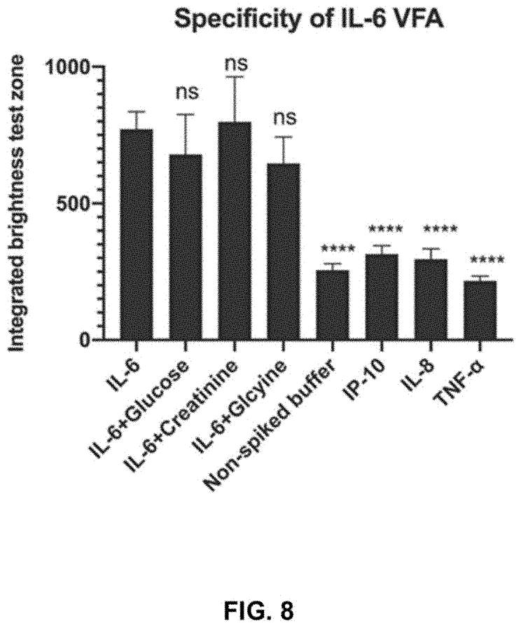

Glucose (1 mg/mL), glycine (30 mg/mL) and creatinine (13.5 μg/mL) were used to evaluate the effect of interferents on rIL-6 detection, while interleukin-8 (IL-8; 200 pg/mL), interferon-gamma inducible protein-10 (IP-10; 200 pg/mL) and TNF-α (200 pg/mL) were used to evaluate the selectivity of the rIL-6 VFA. Addition of glucose, creatinine, and glycine to the rIL-6-piked buffer (50 pg/mL) had no significant changes in the intensity of the test zone measurement compared to the rIL-6-spiked buffer. Furthermore, IP-10, IL-8, and TNF-α spiked buffer showed significantly lower intensities than the rIL-6-spiked buffer, and these intensities were similar to the intensities elicited by the non-spiked buffer. These results, as shown in FIG. 8, indicate that the VFA is specific for the IL-6 detection. All compounds were tested in triplicates.

### Example 6: Use of IL-6 Vertical Flow Assay to Measure Patient Samples

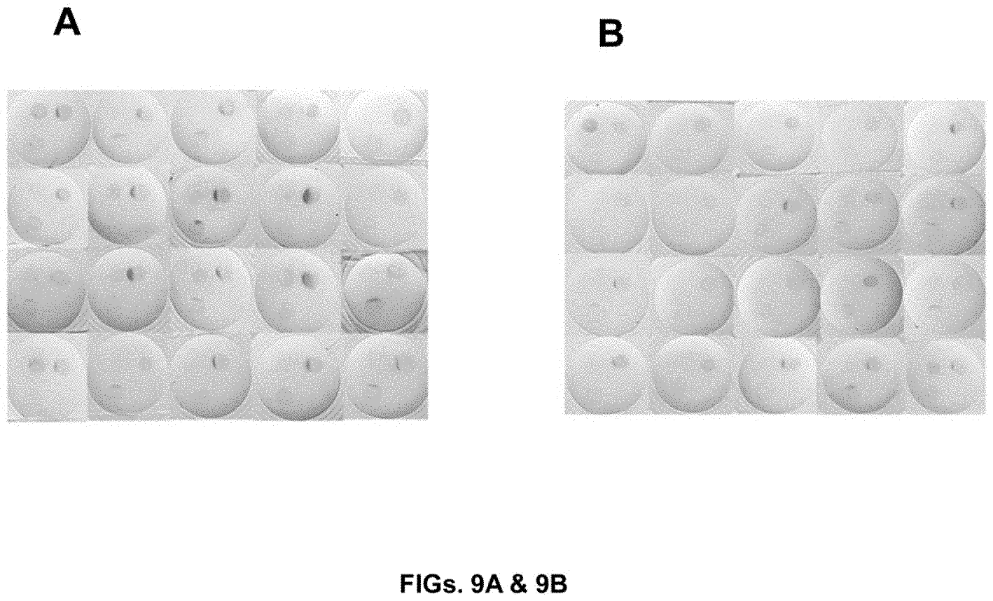

Given that there is a wide range of IL-6 levels in different inflammatory diseases, additional reference zones Std-1 (+3), Std-2 (+2), and Std-3 (+1) were introduced in the VFAs to facilitate semi-quantitative readouts. Std-3 had no biotinylated-BSA (“blank”; bottom right quadrant), while Std-2 (bottom left quadrant) and Std-1 (top right quadrant) had increasing concentrations of biotinylated-BSA spotted at these sites, to serve as concentration indicators. Thus, when streptavidin-conjugated GNP is added, Std-3, Std-2, and Std-1 will show increasing spot intensities in all tests. The IL-6 concentration of Std-3 was blank, that of Std-2 is comparable to the signal from 10-100 pg/ml of IL-6, that of Std-1 is comparable to the signal from 100-1000 pg/ml of IL-6. In this study, 20 RA and 20 HC serum were tested using the IL-6 VFA by following Assay Protocol III (see Methods). As shown in FIGS. 9A and 9B, all VFAs showed varying signals on the TZ (top left quadrant), a strong signal for Std-1 (top right quadrant), an intermediate signal for Std-2 (bottom left quadrant), and no signal on Std-3 (bottom right quadrant). Each subject's IL-6 level after ten-fold dilution was accorded a semi-quantitative observer score (OS), ranging over 0, 1+, 2+, 3+, 4+, and 5+ as detailed in Methods, by three observers, and the imaging scores (IS) of the same 40 samples were recorded (following the method outlined in FIG. 1), in Table 4. Based on the OS, 12 out of 20 RA patients exhibited 2+ or stronger IL-6 readings, while 2 of the 20 healthy subjects exhibited 2+ or stronger IL-6 readings, indicating that RA patients tended to have higher serum IL-6 levels, with the difference attaining significance (Mann Whitney test p-value=0.0043). Table 4 provides the VFA Observer and Imaging Scores.

TABLE 4
IL-6 VFA Observer and Imaging Scores

| ID | OS-1 | OS-2 | OS-3 | OS-Readout | IS-readout |
|---|---|---|---|---|---|
| RA-1 | 4 | 3 | 4 | 4 | 4 |
| RA-2 | 0 | 0 | 0 | 0 | 2 |
| RA-3 | 0 | 0 | 0 | 0 | 1 |
| RA-4 | 2 | 2 | 2 | 2 | 3 |
| RA-5 | 0 | 0 | 0 | 0 | 1 |
| RA-6 | 1 | 1 | 1 | 1 | 1 |
| RA-7 | 3 | 3 | 3 | 3 | 5 |
| RA-8 | 2 | 2 | 2 | 2 | 3 |
| RA-9 | 3 | 2 | 2 | 2 | 3 |
| RA-10 | 2 | 1 | 2 | 2 | 3 |
| RA-11 | 2 | 1 | 1 | 1 | 3 |
| RA-12 | 3 | 2 | 3 | 3 | 3 |
| RA-13 | 5 | 3 | 5 | 5 | 5 |
| RA-14 | 3 | 3 | 3 | 3 | 3 |
| RA-15 | 2 | 1 | 2 | 2 | 1 |
| RA-16 | 4 | 4 | 5 | 4 | 5 |
| RA-17 | 2 | 2 | 2 | 2 | 2 |
| RA-18 | 1 | 1 | 1 | 1 | 2 |
| RA-19 | 1 | 1 | 1 | 1 | 2 |
| RA-20 | 0 | 0 | 0 | 0 | 1 |
| HC-1 | 5 | 5 | 5 | 5 | 5 |
| HC-2 | 0 | 0 | 0 | 0 | 1 |
| HC-3 | 0 | 0 | 0 | 0 | 0.5 |
| HC-4 | 0 | 0 | 0 | 0 | 0.5 |
| HC-5 | 0 | 0 | 0 | 0 | 1 |
| HC-6 | 0 | 0 | 0 | 0 | 1 |
| HC-7 | 0 | 0 | 0 | 0 | 1 |
| HC-8 | 1 | 1 | 1 | 1 | 1 |
| HC-9 | 2 | 1 | 1 | 1 | 2 |
| HC-10 | 1 | 1 | 2 | 1 | 4 |
| HC-11 | 1 | 0 | 1 | 1 | 3 |
| HC-12 | 0 | 0 | 0 | 0 | 0 |
| HC-13 | 0 | 0 | 0 | 0 | 0 |
| HC-14 | 0 | 0 | 0 | 0 | 0.5 |
| HC-15 | 1 | 0 | 1 | 1 | 1 |
| HC-16 | 1 | 1 | 1 | 1 | 1 |
| HC-17 | 0 | 1 | 0 | 0 | 0.5 |
| HC-18 | 0 | 0 | 0 | 0 | 1 |
| HC-19 | 0 | 0 | 0 | 0 | 0 |
| HC-20 | 5 | 5 | 5 | 5 | 5 |

### Materials and Methods

### Materials

The rapid vertical flow assay's plastic housing was purchased from Cytodiagnostics. Seven different makes of nitrocellulose membranes were purchased for membrane screening: Cytiva (Amersham 0.45 um, 0.2 μm and 0.1 μm pore size); BioRad (0.45 μm pore size); MDI (0.8 um, 0.45 um, and 0.3 μm pore size); Absorbent pads were purchased from Cytiva (CF7 and CF5); Polyethylene glycol (PEG 3350), polyvinylpyrrolidone (PVP40), bovine serum albumin (BSA), Tris-HCl, phosphate buffer saline (PBS, pH 7.4), Ethylenediaminetetraacetic acid (EDTA), gelatin and sodium chloride (NaCl) were purchased from Sigma Aldrich. Tween-20 was purchased from Promega Corporation. Creatinine, recombinant interleukin-8, interferon-gamma inducible protein-10 (IP-10), and TNF-α were purchased from R&D Systems (Minneapolis, Minn., USA) for specificity evaluation. The anti-IL-6 capture antibody, recombinant IL-6 protein, biotinylated anti-IL-6 detection. The anti-IL-6 capture antibody, recombinant IL-6 protein, and biotinylated anti-IL-6 detection antibody were purchased from R&D Systems, as well as the human IL-6 duoset ELISA kit. 150 nm and 40 nm streptavidin-conjugated GNP were purchased from Nanocomposix.

### Patients and Samples Collection

Twenty (20) healthy control (HC) serum samples were obtained from the UT Southwestern Medical Center, with informed consent (Dallas, Tex., USA). The 20 rheumatoid arthritis (RA) serum samples were purchased from BioIVT (New York, USA). Serum samples were aliquoted and stored at −20° C. Before use, thawed serum samples were centrifuged at 5000 rpm for 5 min to remove any lipids or debris, and only the clear serum was applied to the assay.

### Assay Protocol I: IL-6 Detection in Ten-Fold Diluted Healthy Serum

For the detection of IL-6 in serum, pooled healthy serum was first ten-fold diluted in serum diluent (9 mM Tris-HCl, 67.5 mM NaCl, 1.5 mM EDTA, 1.5% BSA, 0.12% Tween-20, 0.05% gelatin, 0.25% PEG, pH 8, filtered), and then spiked with recombinant IL-6; 100 pg/ml was used as positive control; the non-spiked serum was used as a negative control. It was found that using an absorbent pad of size 2.2 cm×2.7 cm and a membrane of size 1.5 cm×1.5 cm yielded a clear signal with minimal GNP aggregation. VFAs were first assembled by clamping two layers of NCM at the top, and one layer of CF7, and two layers of CF5 at the bottom within the plastic housing. The following protocol was adapted for preliminary screening of membrane pore size. 1 μL of 1 mg/ml of anti-IL-6 capture antibody in PBS was spotted at the top left quadrant of the nitrocellulose membrane (NCM). The NCM was air blown for one hour. After that, the cartridge was stored at room temperature with desiccator pouches until needed. To run the VFA, the NCM was washed using 100 μL of the washing buffer (20 mM Tris-HCl, 150 mM NaCl, 1% BSA, 0.1% Tween-20, pH 8, filtered). Then, 200 μL of blocking reagent (3 mM EDTA, 3% BSA, 0.1% gelatin, filtered) was applied to the NCM for 5 min. Following an additional 100 μL of the washing buffer, 100 μL of 10-fold diluted serum sample (positive control: final concentration 100 pg/ml) as prepared above was loaded to the top left quadrant of the NCM. Following additional 100 μL of the washing buffer, 10 μL of 10 pg/mL biotinylated anti-IL-6 detection antibody in GNP diluent (20 mM Tris-HCl, 150 mM NaCl, 0.5% PVP40, 1% BSA, 0.19% Tween-20, pH 8, filtered) was loaded into the top left quadrant of the NCM. Following additional 100 μL of the washing buffer, 10 μL of 40 nm streptavidin-conjugated GNP (SA-GNP) (OD=5) in the GNP diluent was loaded into the top left quadrant. After 30 seconds, 400 μL of washing buffer was applied to remove any background.

### Assay Protocol II: IL-6 Detection in Spiked Buffer and Spiked Pooled Healthy Serum for the Evaluation of Detection Limit

Certain steps of this protocol are illustrated with respect to FIG. 3B. For the detection of IL-6 in buffer, recombinant IL-6 was spiked to the serum diluent to constitute 0, 10, 100, 1000, 10,000 pg/m<concentrations. For the detection of IL-6 in serum, pooled healthy serum was first ten-fold diluted in serum diluent and spiked with recombinant IL-6 to constitute 0, 1, 3.2, 10, 32, 50, 100, 200, 320, 400, 600, 800, 1000, 3200, and 10,000 pg/ml concentrations. The VFAs were first assembled by clamping two layers of Amersham 0.2 μm NCM at the top, and one layer of CF7 and two layers of CF5 at the bottom within the plastic housing. The assay protocol is as follows. One (1) μL of 1 mg/ml anti-IL-6 capture antibody in PBS was spotted at the top left quadrant of the NCM. The NCM was air blown for one hour. After that, the cartridge was stored at room temperature with desiccator pouches until needed. To run the VFA, the NCM was washed using 100 μL of the washing buffer (20 mM Tris-HCl, 150 mM NaCl, 1% BSA, 0.1% Tween-20, pH8, filtered). Then, 200 μL of blocking reagent (3 mM EDTA, 3% BSA, 0.1% gelatin, filtered) was applied to the NCM for 5 min. Following additional 100 μL of washing buffer, 500 μL of samples (buffer or ten-fold diluted serum) as prepared above was loaded onto the entire NCM. After absorbing excess sample, 100 μL washing buffer was applied, followed by 20 μL 10 μg/mL of biotinylated anti-IL-6 detection antibody in GNP diluent (9 mM Tris-HCl, 67.5 mM NaCl, 0.5% PVP40, 1% BSA, 0.19% Tween-20, pH 8, filtered), loaded to the top left quadrant of the NCM for 30 seconds. Following additional 100 μL of the washing buffer, 20 μL of 40 nm streptavidin-conjugated GNP (OD=5) in GNP diluent was loaded to the top left quadrant. After 30 seconds, 400 μL of washing buffer was applied to remove any background.

### Assay Protocol III: IL-6 Detection in Patient Samples and Healthy Serum

To assay the IL-6 level in 20 rheumatoid arthritis (RA) and 20 healthy control (HC) serum samples, 1 μL of 0.5 mg/ml anti-IL-6 capture antibody in PBS was spotted at the top left quadrant, referred as the test zone; 1 μL of 0.5 mg/mL biotinylated-BSA in PBS, 5% ethanol was spotted at the top right quadrant, referred as Std-1 (+3); 1 μL of 0.1 mg/mL biotinylated-BSA in PBS, 5% ethanol was spotted at the lower left referred as Std-2 (+2); 1 μL of PBS was spotted at the lower right quadrant, referred as Std-3 (+1). Protocol-III is similar to Protocol-II, except for the following changes. After loading 20 μL streptavidin-conjugated GNP in GNP diluent to the top left for 30 seconds, 200 μL of washing buffer was applied. Then, 0.5 μL of 40 nm streptavidin-conjugated GNP (OD=5) in GNP diluent was added to the top right quadrant, 0.5 μL to the lower left quadrant, and 0.5 μL to the lower right quadrant. After 10 seconds, 200 μL of washing buffer was loaded to remove any background. The semiquantitative recording of IL-6 levels in the clinical samples followed the following criteria: if the signal intensity at test zone is higher than Std-3 but lower than Std-2, it is recorded as +1; if the signal intensity is similar to Std-2, it is recorded as +2; if the signal intensity is higher than Std-2 but lower than Std-1, it is recorded as +3; if the signal intensity is higher than Std-1 the sample is recorded as +4; and if the signal intensity is higher than all 3 standards, OS is recorded as “+5”, in illustrated in FIG. 3C.

The algorithm used for assigning the semi-quantitative scores corresponding to the smartphone-reported imaging score (IS) was identical to the algorithm described above for the OS, with one exception. For each absolute value captured by the phone at the test zone (TZ), 15% coefficient of variation was allowed. Thus, for example, the IS reported as “+1” if 0.85×TZ and 1.15×TZ were both higher than Std-3 intensity but lower than Std-2; it is recorded as “+2” if 0.85×TZ was lower than Std-2 but 1.15×TZ was greater than Std-2; recorded as “+3” if 0.85×TZ and 1.15×TZ were both higher than Std-2 intensity but lower than Std-1 intensity. Similar reporting was used across all included standards, not unlike the algorithm used for interpreting the OS.

### Assay Protocol IV: Further Evaluation of Characteristics and Performance

In Assay Protocol IV, in total, 80 μL of buffer-1 (20 mM Tris, 150 mM NaCl, 1% BSA, 0.18% Tween-20, 0.5% PEG, pH 8, filtered) was applied to the nitrocellulose membrane (NCM), and then 80 μL of blocking buffer (3 mM EDTA, 3% BSA, 0.1% gelatin, filtered) was added to NCM for 5 min. Following an additional 80 μL wash with buffer-1, 100 μL of buffer-1 spiked with the analyte or non-spiked (0 ng/mL) was loaded onto two spotted quadrants, respectively. Following an 80 μL wash with buffer-1, 20 μL of 10 μg/mL biotinylated anti-IL-6 in buffer-1 was individually loaded to the two quadrants in NCM. Following an additional 80 μL wash with buffer-1, 5 μL of streptavidin-conjugated GNP (OD=2) in buffer-1 was loaded to the two quadrants, respectively. After 30 s, 800 μL of buffer-1 was loaded to wash off any background.

### Assay Protocol V: Further Evaluation of Characteristics and Performance

In Assay Protocol V, buffer-2 (20 mM Tris, 150 mM NaCl, 1% BSA, 0.18% Tween-20, 0.5% PVP40, pH 8, filtered) was substituted for buffer-1 in Assay Protocol IV and the remaining steps were unchanged.

### Data Analysis

Images of the cartridges were captured using an iScanner app in iPhone 12. The images were first inspected by naked eye and then analyzed by Image J, by placing a line width of 8 pixels across the center of the test spot where the scale is 1 pixel/unit ratio. The dose-response curves and the biomarker data from VFA and commercial ELISA were plotted and analyzed using GraphPad Prism 5 (GraphPad, San Diego, Calif., USA). Biomarker group comparisons of VFA and commercial ELISA were analyzed using the Mann-Whitney U-test as datasets were not normally distributed. The one-way ANOVA non-parametric test was used to analyze the LoD and linearity (R²) metrics derived for VFA stability evaluation. The LoD was the lowest concentration that could be detected, represented by the sum of the mean of the blanks (n=2) plus three times the standard deviation of the blanks. Receiver operating characteristic (ROC) curves were plotted and the area under the curve (AUC) was used as a measure of the discriminative power of the assay.

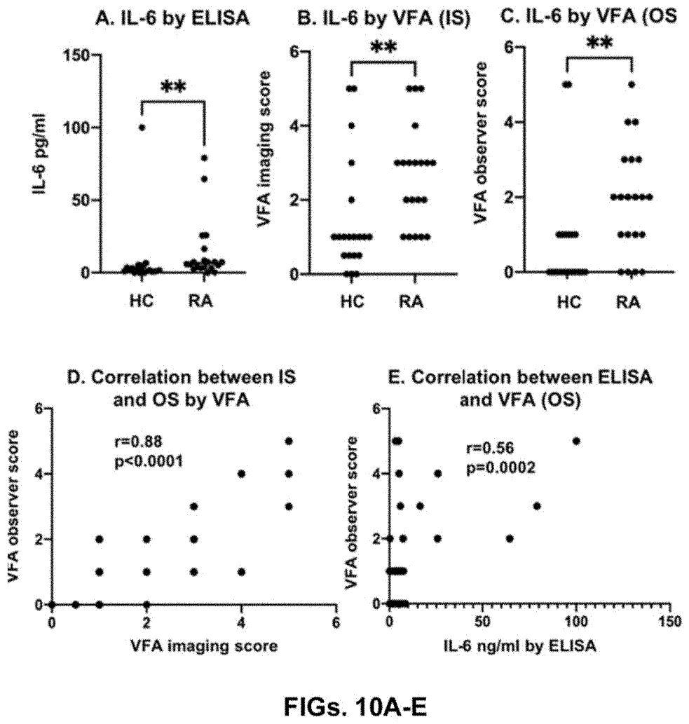

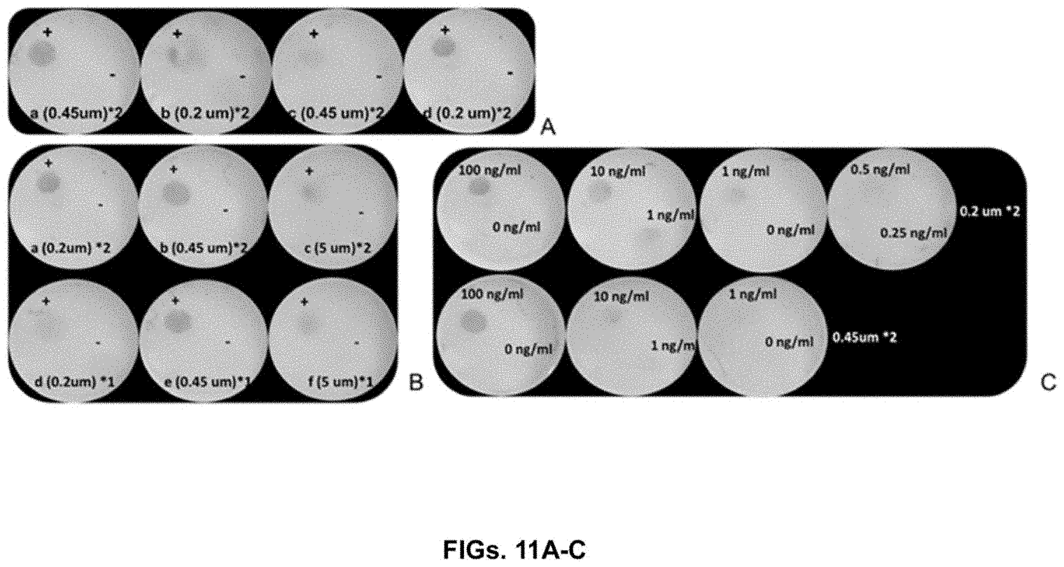

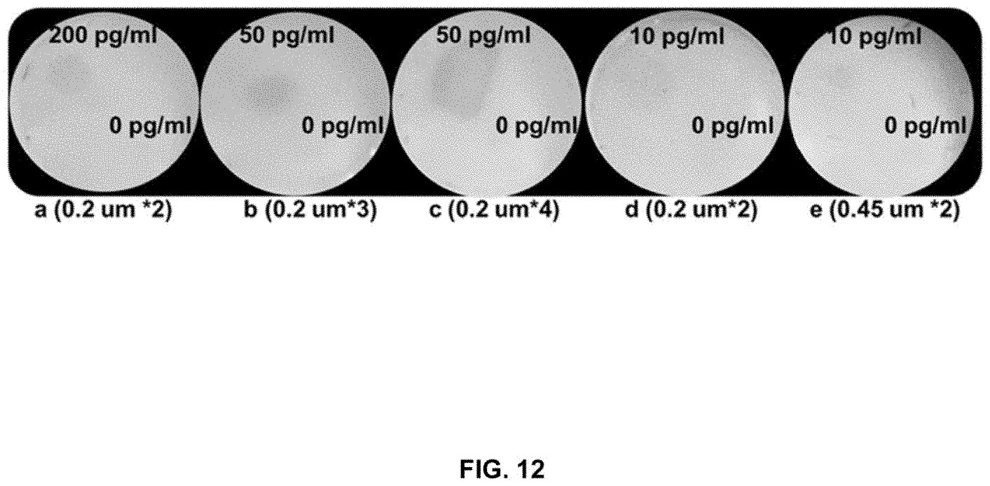

The same serum samples from 20 RA patients and 20 HC subjects tested using the IL-6 VFA were also evaluated using a commercial ELISA for IL-6. The IL-6 levels in RA patients showed a significantly higher value than HC (** p<0.01, FIG. 10A), as did the imaging scores (IS) and observer scores (OS) for the IL-6 VFA (** p<0.01, FIGS. 10B, 0C). As shown in FIG. 10D, a strong correlation between the IS and OS IL-6 scores was observed (a Pearson correlation of 0.88). The correlation between the IL-6 levels assayed by ELISA and VFA was modest (a Spearman correlation of 0.56), as displayed in FIG. 10E.

VFA is a valuable tool for detecting IL-6 in serum. The VFA platform disclosed herein is quick, efficient, and on par with ELISA in terms of accuracy and sensitivity, particularly given that ELISA is a procedure that takes hours to days to complete. Furthermore, the VFA has a low cost, a fast response time, and is extremely easy to use. This makes it advantageous over other serum IL-6 detection approaches, as well as for assaying other blood cytokines. The LoD of rIL-6 in spiked serum was determined to be 3.2 pg/mL, with a wide reportable range of up to 10,000 pg/mL of rIL-6. Thus, the presently engineered VFA is able to detect IL-6 in most of the inflammatory diseases, allowing early detection of a rise in IL-6. Detecting an early rise in IL-6 levels could be of clinical use in triaging patients for admission to hospital/ICU, for commencing IL-6 targeted therapeutics, or for assessing disease activity in multiple life-threatening diseases.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A vertical flow assay device for detecting presence or an amount of interleukin 6 (IL-6) in a sample, the device comprising:
a membrane member having a first surface and a second surface;
a capture agent immobilized on or within the membrane member, wherein the capture agent binds to IL-6;
an absorbent pad member having a first surface and a second surface, positioned under the membrane member, wherein the second surface of the membrane member and the first surface of the absorbent pad member are in contact with each other; and
a holder with a first wall and a second wall, wherein:
the first wall is positioned proximal to the first surface of the membrane member;
the first wall has an opening traversing the first wall, the opening exposing a portion of the first surface of the membrane member to outside of the vertical flow assay device;
the second wall is positioned under the absorbent pad member; and
the holder provides a tight-fit chamber in which the membrane member and the absorbent pad member are contained.

2. The vertical flow assay device of claim 1, wherein the membrane member comprises one or more porous solid state materials selected from one or more of plastic, polymers, paper, nitrocellulose, cellulose, PVDF, polycarbonate, ceramic, metallic materials, glass, glass microfibers, and anodized aluminum.

3. The vertical flow assay device of claim 1, wherein the membrane member comprises a nitrocellulose membrane.

4. The vertical flow assay device of claim 1, wherein the membrane member contains pores of 10 μm or less in size and has a porosity of between 10% and 95%.

5. The vertical flow assay device of claim 1, wherein the membrane member comprises pores that are about 0.2 μm in size.

6. The vertical flow assay device of claim 1, wherein the absorbent pad member comprises one or more absorbent pads.

7. The vertical flow assay device of claim 6, wherein the absorbent pad member contains pores ranging from between 1 μm to 1000 μm in size, and has thickness of between 1 mm to 10 mm at 53 kPa.

8. The vertical flow assay device of claim 1, wherein the absorbent pad member comprises one or more absorbent pads made of cotton linter material.

9. The vertical flow assay device of claim 1, wherein the holder comprises a standard fitting configured to mount onto a detection system, and a portion of the membrane member can be accessed through the opening of the first wall of the holder to provide a detectable signal indicative of the presence of the amount of IL-6 to the detection system.

10. The vertical flow assay device of claim 9, wherein the detection system is an imager, a scanner, or a smartphone.

11. The vertical flow assay device of claim 1, wherein the sample is a body fluid sample.

12. The vertical flow assay device of claim 11, wherein the sample is a serum sample.

13. The vertical flow assay device of claim 1, wherein the device is disposable.

14. An immunoassay method for detecting presence or an amount of interleukin 6 (IL-6) in a sample, comprising:
providing the vertical flow assay device of claim 1;
applying a sample onto the membrane member through the opening of the first wall of the holder;
applying a detection agent that binds to IL-6 present in the sample, thereby forming a sandwich complex comprising the capture agent, IL-6, and the detection agent;
providing a detectable signal responsive to presence or an amount of IL-6 in the sample bound in the sandwich complex; and
relating the detectable signal to the presence or the amount of IL-6 in the sample.

15. The immunoassay method of claim 14, wherein a reportable range of IL-6 in the sample is from about 10 pg/ml to about 1,000 pg/ml.

16. The immunoassay method of claim 14, wherein the vertical flow assay device contains a positive control spot and/or a negative control spot on the membrane member.

17. The immunoassay method of claim 14, wherein the capture agent and/or the detection agent is an antibody that binds to IL-6.

18. The immunoassay method of claim 14, comprising:
mounting the vertical flow assay device to a detection system to provide the detectable signal, and
determining the presence or amount of IL-6 in the sample.

19. The immunoassay method of claim 18, wherein the detection system is an imager, a scanner, or a smartphone.

20. A kit for detecting presence or an amount of interleukin 6 (IL-6) in a sample, comprising:
a vertical flow assay device of claim 1;
a capture reagent and a detection reagent that each bind to IL-6;
a signal development element that can bind to the detection reagent;
a washing buffer;
a blocking buffer;
a calibration marker to relate a signal to the presence or the amount of IL-6; and/or
an instruction for use.
